(19) United States
(12) Patent Application Publication
(10) Pub. No.: US 2025/0345576 A1
(43) Pub. Date: Nov. 13, 2025

(54) PERCUTANEOUS DRAIN SECUREMENT DEVICE AND METHOD

(71) Applicant: Northwestern University, Evanston, IL (US)

(72) Inventors: Ahsun RIAZ, Evanston, IL (US); Gabriel KNIGHT, Evanston, IL (US); Abhinav TALWAR, Evanston, IL (US); Riad SALEM, Evanston, IL (US)

(21) Appl. No.: 19/203,928

(22) Filed: May 9, 2025

Related U.S. Application Data
(60) Provisional application No. 63/645,624, filed on May 10, 2024.

(51) Int. Cl. A61M 27/00; A61F 13/02; A61F 15/00

(52) U.S. Cl. CPC A61M 27/00; A61F 13/024; A61F 15/005; A61M 2205/582

(57) ABSTRACT

Provided herein, is a device and method for fixing a percutaneous drain to a patient's skin while allowing for easy dressing changes. The device may comprises a drain engagement element with a cylindrical lumen for receiving the drain and two horizontal struts extending from its top portion, configured to snap into hooks on a back plate adhered to the patient's skin, creating a suture-less “air knot” that fixes the drain in place while allowing rotation in one plane to maintain a fixed curvilinear distance between the drain incision site and the drain engagement element attachment site. This arrangement minimizes drain movement and kinking during normal patient activities. A dressing change tool with hooks aligned to those on the back plate allows the drain engagement element to be temporarily transferred to the tool during dressing changes, maintaining the drain's position and avoiding dislodgement. The device reduces patient discomfort and complications associated with traditional drain fixation methods.

### FIELD

Provided herein are devices and methods for sutureless securement of medical devices to a subject. In particular, drain tubes (e.g., percutaneous drains), catheters, and other medical devices are secured to the skin of a patient (e.g., near the insertion site) using the devices and methods herein.

### BACKGROUND

Clinicians, for example, interventional radiologists (IR) and surgeons, place drains and other catheters percutaneously as conduits for fluid to be removed from bodily cavities. A growing body of evidence indicates that complications of drain placement-pain, leakage, dislodgment, and infection-occur frequently. Many of these complications are attributable, at least in part, to the methods used to place and secure drains. Suture is often used to tie drains to skin, but loose knots predispose to drain dislodgment while tight knots frequently cause pain and serve as niduses for infection. Adhesive drain fixation devices lose efficacy over time and make dressing changes unnecessarily complicated. What is needed is a device that allows for stable and secure attachment of drain/catheter devices, while allowing for patient movement and dressing changes, without producing discomfort of adverse events.

### SUMMARY

Provided herein are devices and methods for sutureless securement of medical devices to a subject. In particular, drain tubes (e.g., percutaneous drains), catheters, and other medical devices are secured to the skin of a patient (e.g., near the insertion site) using the devices herein.

In some embodiments, provided herein are drain securement devices comprising: drain engagement element comprising: a cylindrical lumen running vertically through the drain engagement element and adapted to receive a drain, and a pair of horizontal struts extending from a top portion of the drain engagement element; and a back plate comprising: an adhesive material on a posterior surface adapted to adhere to a patient's skin; and a pair of hooks configured to releasably engage the horizontal struts of the clamp; wherein, when the horizontal struts are engaged with the hooks, the clamp is fixed to the patient's skin while allowing rotation of the clamp in one plane to maintain a fixed curvilinear distance between a drain incision site and a drain-clamp attachment site.

In some embodiments, the drain engagement element comprises a front half and a back half that are configured to engage each other to form the cylindrical lumen.

In some embodiments, the front half and a back half are joined on one side by a hinge.

In some embodiments, the drain engagement element further comprises a protrusion extending into the lumen, wherein the protrusions sized and configured to clamp the drain without occluding the lumen; and

In some embodiments, the horizontal struts and the hooks are configured to permit engagement and disengagement of the clamp with the back plate in only one orientation.

In some embodiments, when the horizontal struts are engaged with the hooks, the drain engagement element allows for minimal longitudinal movement of the drain while maintaining the drain in a fixed position relative to the patient's skin.

In some embodiments, the drain securement device further comprises a dressing change tool comprising: an adhesive material on a posterior surface adapted to adhere to the patient's skin; and a pair of hooks configured to releasably engage the horizontal struts of the drain engagement element.

In some embodiments, the hooks of the dressing change tool are aligned with the hooks of the back plate when the dressing change tool is adhered to the patient's skin above the back plate.

In some embodiments, the hinge is a book flap hinge.

Provided herein are methods of changing a dressing on a percutaneous drain fixed to a patient's skin, the methods comprising: adhering a dressing change tool to the patient's skin above a back plate including a pair of hooks, the dressing change tool comprising: an adhesive material on a posterior surface; and a pair of hooks on an anterior surface, the hooks aligned with the hooks of the back plate; disengaging the horizontal struts of a drain engagement element from the hooks of the back plate and engaging the horizontal struts with the hooks of the dressing change tool; removing the back plate from the patient's skin; adhering a new back plate to the patient's skin; disengaging the horizontal struts of the drain engagement element from the hooks of the dressing change tool and engaging the horizontal struts with hooks of the new back plate; and removing the dressing change tool from the patient's skin; wherein the fixed curvilinear distance between the drain incision site and the drain engagement element attachment site is maintained while the horizontal struts are engaged with the hooks of the dressing tool.

In some embodiments, the percutaneous drain is selected from the group consisting of a Jackson-Pratt drain, a Blake drain, a Penrose drain, and a pigtail catheter.

In some embodiments, the percutaneous drain is adapted to drain fluid from a body cavity selected from the group consisting of a pleural space, a peritoneal cavity, and a retroperitoneal space.

In some embodiments, securing the percutaneous drain with the drain securement device reduces at least one complication selected from the group consisting of drain dislodgement, drain kinking, and drain occlusion, as compared to securing the drain with sutures or adhesive alone.

In some embodiments, the horizontal struts comprise triangular grooves formed on outer surfaces of the struts.

In some embodiments, the hooks of the back plate comprise triangular rails protruding inwardly from inner surfaces of the hooks, the triangular rails configured to mate with the triangular grooves of the horizontal struts.

In some embodiments, the triangular grooves and triangular rails form a keyed connection that permits engagement and disengagement of the drain engagement element with the back plate only when the triangular grooves are aligned with the triangular rails.

In some embodiments, the keyed connection provides tactile feedback to confirm proper connection between the drain engagement element and the back plate.

In some embodiments, the keyed connection reduces risk of accidental decoupling during patient movement.

In some embodiments, the horizontal struts comprise triangular grooves formed on outer surfaces of the struts, the hooks of the back plate comprise first triangular rails protruding inwardly from inner surfaces of the hooks, and the hooks of the dressing change tool comprise second triangular rails protruding inwardly from inner surfaces of the hooks, wherein both the first and second triangular rails are configured to mate with the triangular grooves of the horizontal struts.

Provided herein are drain securement devices comprising a drain engagement element comprising a lumen adapted to receive a drain, and at least one horizontal strut extending from the drain engagement element, the at least one horizontal strut having a triangular groove formed on an outer surface, a back plate comprising an adhesive material on a posterior surface adapted to adhere to a patient's skin, and at least one hook configured to releasably engage the at least one horizontal strut, the at least one hook having a triangular rail protruding inwardly from an inner surface configured to mate with the triangular groove of the at least one horizontal strut, wherein the triangular groove and triangular rail form a keyed connection that permits engagement and disengagement of the drain engagement element with the back plate only when the triangular groove is aligned with the triangular rail.

### BRIEF DESCRIPTION OF THE DRAWINGS

FIG. 1. Views of a securement device according to one embodiment of the present disclosure are provided, including a clamp assembled around a drain and a black plate.

FIG. 2. Views of the clamp and back plate being secured to each other via hooks and struts at a specific orientation are provided according to one embodiment of the present disclosure.

FIG. 3. Views of the clamp rotating in a fixed plane such that there is a fixed curvilinear distance between the drain incision site and the drain-clamp attachment site is provided according to one embodiment of the present disclosure.

FIG. 4. Views of a securement device allowing for minimal in-and-out movement of the drain while keeping the drain firmly fixated to skin thereby creating a suture-less air knot according to one embodiment of the present disclosure are provided.

FIG. 5. Views of the steps involved in operating a device and changing an existing dressing are provided according to one exemplary method of the present disclosure.

FIG. 6. A view of a drain engagement element according to another embodiment of the present disclosure is provided, including triangular grooves formed in the horizontal struts.

FIG. 7. A view of a backplate according to another embodiment of the present disclosure is provided, including triangular rail features.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “subject” broadly refers to any animal, including but not limited to, human and non-human animals (e.g., dogs, cats, cows, horses, sheep, poultry, fish, crustaceans, etc.). As used herein, the term “patient” typically refers to a subject that is being treated for a disease or condition.

### DETAILED DESCRIPTION

Provided herein are devices for sutureless securement of medical devices to a subject. In particular, drain tubes (e.g., percutaneous drains), catheters, and other medical devices are secured to the skin of a patient (e.g., near the insertion site) using the devices herein. Provided herein are medical devices that function to fix a medical line (e.g., catheter, percutaneous drain, IV tubing, etc.) to a patient without the use of a suture. In some embodiments, the devices herein allow the medical line to be stably held in place with respect to the point of entry into the subject (e.g., an incision or orifice). In some embodiments, a device allows the patient to move while the line is secured by the device (e.g., without disrupting the insertion of the line). In some embodiments, a device allows the position of the body of the medical line (not the insertion stie) to be adjusted while the line is secured by the device. Certain embodiments are described herein for use with percutaneous drains; however, the embodiments described herein may also find use in the sutureless securement of other medical lines, such as catheters (e.g., urinary catheters) and IV tubing. Embodiments herein are not limited by the identity of the medical lines to be used therewith.

In some embodiments, the devices herein are sutureless drain fixation devices that are more comfortable for patients, easier to exchange during dressing changes, and decrease the incidence of drain-related adverse events. In some embodiments, the drain securement devices herein do not evert or tug at skin which provides increased comfort for patients. In some embodiments, securement devices herein apply circumferential pressure around the site of insertion (e.g., drain) to provide strong fixation, decreased risk of dislodgment, and decreased risk of kinking/obstruction. In some embodiments, because the tip of the drain can move freely in one plane, the devices herein provide decreased intra-corporeal “play” (i.e., the end of the drain in the body moves less; is more stable) when the outside tip is manipulated. In some embodiments, devices herein decrease the risk of dislodgment compared to existing techniques. In some embodiments, because the tubing does not adhere to the device, the device allows for quicker and easier changing of the drain, dressing, etc. In some embodiments, the incision site is not exposed to suture which can predispose to infection. Therefore, the devices herein decrease the risk of infection.

In particular embodiments, provided herein are devices for securing a medical line (e.g., tube) at an incision site of a patient. In some embodiments, the medical line is a percutaneous drain. Exemplary medical lines that find use with embodiments of the devices herein may include an endotracheal tube, a tracheotomy tube, chest tube, a vascular catheter, a urinary catheter, a nephrostomy tube, a biliary stent, a peritoneal catheter, an epidural catheter, a central nervous system catheter, a prosthetic valve, a gastric tube, an intestinal tube, drug-delivery implant, or an intravenous tube. In some embodiments, the devices herein find use in securing any medical line that enters the subject (e.g., at an incision or through an orifice) and need to be held in place relative to the entry site and/or on the skin of the subject.

In some embodiments, the device of the present disclosure is created using three components: a clamp which, in some embodiments, may snap tightly around the percutaneous drain and hang from the hooks of the back plate, a back plate which, in some embodiments, covers the incision site and includes hooks that allow for the clamp to hang from, and a dressing change tool which, in some embodiments, temporarily adheres to the patient's skin above the back plate at the time of a dressing change and includes hooks that allow for the clamp to attach to the back plate. In alternative embodiments, the device may include fewer or additional components. For example, the device may only include a clamp and back plate, with the dressing change tool being optional or separately provided.

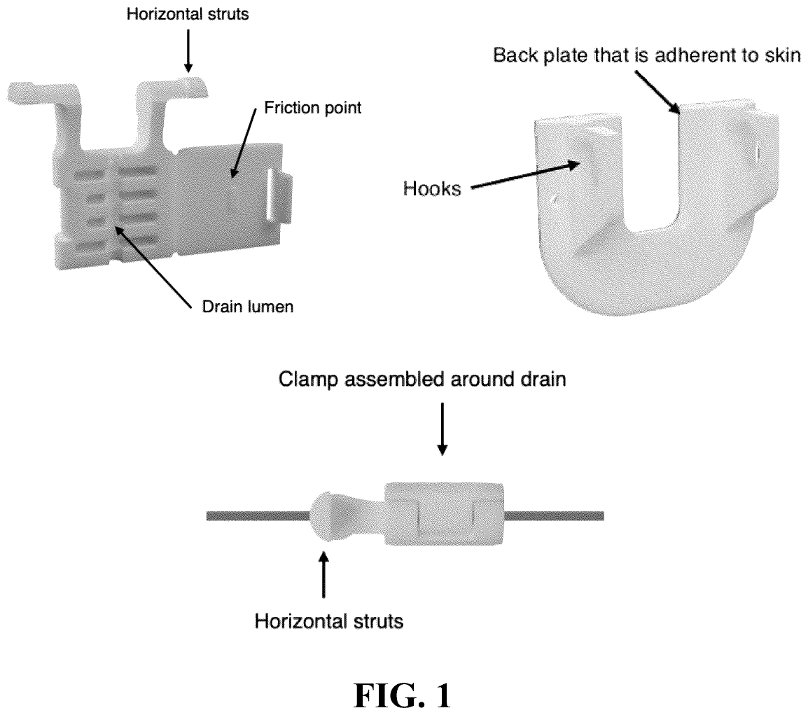

Referring to FIG. 1, in some embodiments, the clamp may be composed of a front and back half that are joined via a “book flap” hinge. In alternative embodiments, the clamp may comprise two separate parts that clip, snap, slide, screw, or otherwise reversibly connect together around the drain tube, rather than being permanently connected by a hinge. For example, the clamp halves may have interlocking tab and slot features, or a pin and hole that allow them to snap together. The clamp halves may be identical or may have complementary mating features. In further alternatives, the clamp may include three or more pieces that assemble together around the drain tube, such as a clamshell design with a top half, bottom half, and hinge piece.

In some embodiments, the back half of a hinged clamp, or a portion of a non-hinged clamp, may contain a cylindrical lumen where the percutaneous drain is to be placed. In some embodiments, the lumen may be located in the front half of the clamp, in a middle portion between front and back halves, or split between the front and back halves. In some embodiments, the lumen may have a non-cylindrical cross-section, such as an oval, polygon, or irregular shape, to match the cross-section of the drain tube. A long the lumen there may be one or more small protrusions which allow the device to clamp down on the drain without fully occluding it. These protrusions may have a rounded, pointed, or textured surface. In some embodiments, the protrusions may be on opposing sides of the lumen to grip the drain tube, for example one protrusion on each half of a clamshell clamp. In other alternatives, the lumen may include multiple protrusions projecting inwardly from around its circumference to hold the drain tube. These protrusions may be arranged in a circumferential array, in a spiral pattern, or in linear rows.

In some embodiments, the top of the clamp forms two horizontal struts protruding from either side. These struts may be cylindrical protrusions, square tabs, rectangular bars, or have another shape. In some embodiments, these struts may be inserted into the back plate to effectively fix the drain in place. In alternative embodiments, the clamp may have a different number of struts, such as one, three, four, or more. In other alternatives, the clamp may have hook, loop, eyelet, or clip structures instead of struts to engage the back plate and/or dressing. In some other embodiments, the attachment mechanisms may be reversed, with the hooks, struts or other connectors located on the back plate and/or dressing change tool, and the receiving holes or other mating features located on the clamp.

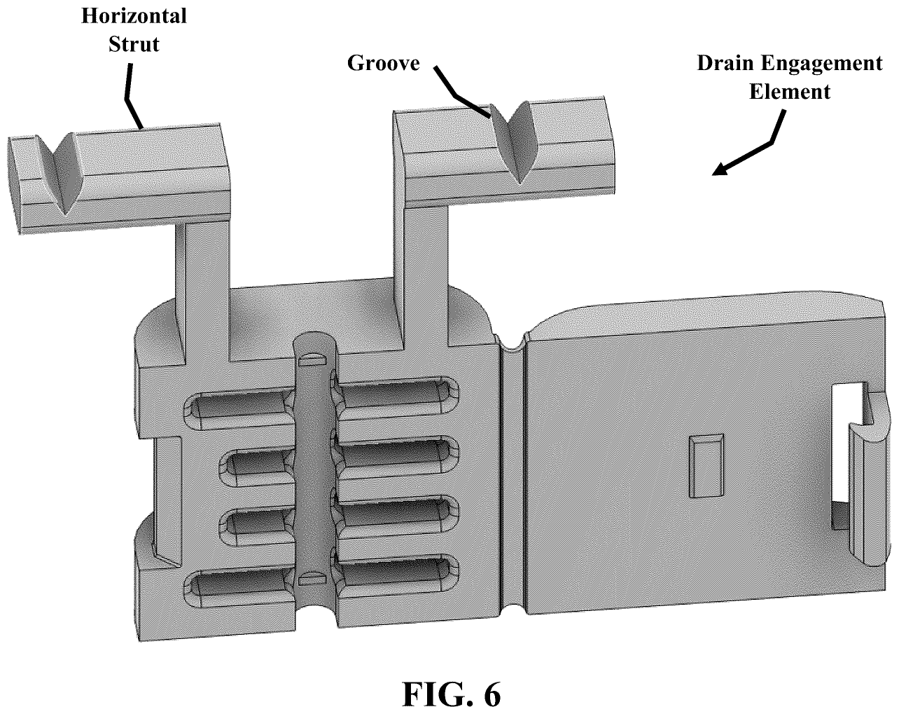

In certain embodiments, as illustrated in FIG. 6, the horizontal struts of the clamp include triangular grooves formed along their outer surfaces. These grooves are sized and configured to mate with corresponding triangular rails on the back plate hooks. The triangular cross-section of both the grooves and rails creates a self-aligning mechanism that guides the struts into proper position during connection. The triangular profile prevents rotation or misalignment once engaged, providing additional stability to the connection between the clamp and back plate.

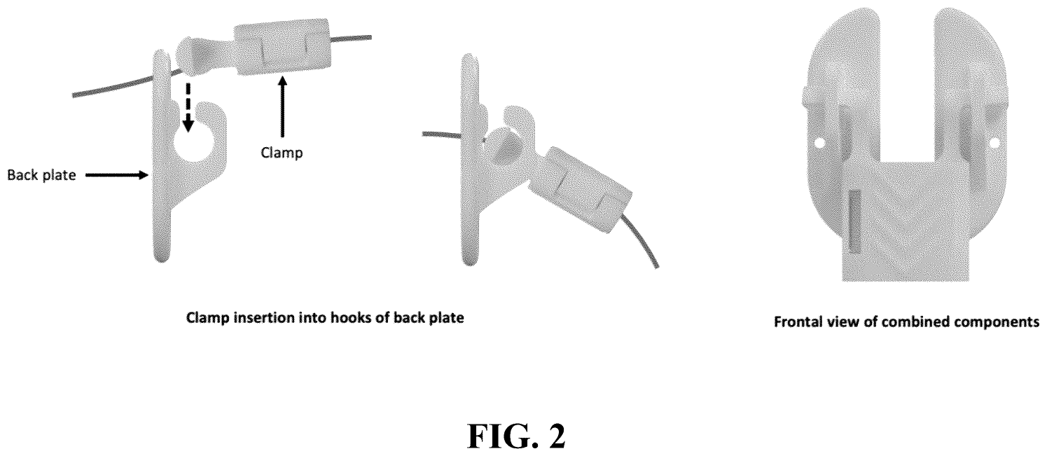

Referring to FIG. 2, the back plate may include an adhesive material on its posterior (skin-abutting) portion and hooks. The hooks may be J-shaped, U-shaped, V-shaped, T-shaped, or have another shape. The hooks may have a narrow neck and wider head to hold the clamp struts in place. The hooks may be made of plastic, metal, or another material. The back plate may have a different number of hooks or other connectors than shown, such as zero, one, three, four or more hooks. In some embodiments, the backing plate's hooks or connectors may be located in a different position, such as on the front surface of the plate rather than protruding from the top edge, or on the sides or bottom of the plate. In other embodiments, the hooks or other connectors may be located on a stem, post, or other structure extending vertically and/or horizontally from the plate. In some embodiments, the back plate may be adhered to the patient's skin around the drain incision site using the adhesive material. The hooks may have openings facing opposite directions. In the presently pictured embodiment, the back plate includes two hooks that extend orthogonally from the backplate.

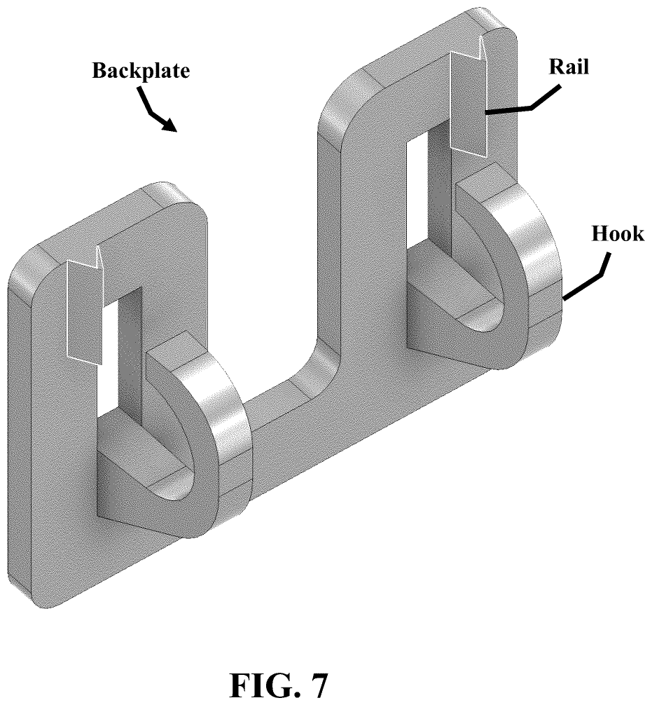

Referring to FIG. 7, in some embodiments, the back plate hooks include triangular rails that protrude inwardly from the inner surface of each hook. These triangular rails are sized and configured to mate with corresponding triangular grooves on the clamp's horizontal struts. The triangular profile of the rails ensures that the clamp can only be inserted or removed from the back plate in one specific orientation, i.e. when the grooves are perfectly aligned with the rails. This configuration significantly reduces the risk of accidental decoupling of the clamp from the back plate during patient movement or drain manipulation, thereby enhancing the security and stability of the drain fixation.

In some embodiments, the horizontal struts formed in the securement device are sized and configured to snap into the hooks on the back plate as shown in FIG. 2. The struts may have bulbous or flared ends to engage the hooks. The strut ends and hook openings may be complementary in size and shape to allow them to fit together. In some embodiments, the struts are sized and configured in such a way that only one orientation of the clamp will allow for the hooks and struts to snap together. For example, the struts may be different sizes or different distances apart to enforce a particular orientation. In the presently disclosed embodiment, the hooks are generally circular shape with a missing segment. Such segment is sized and configured to only accept the corresponding shape of the strut is a single orientation do the maximum allowed space between the two ends of the hook segment and the minimum thickness of the strut in that particular orientation. Similarly, in some embodiments, only one orientation of the clamp will allow for the components to separate.

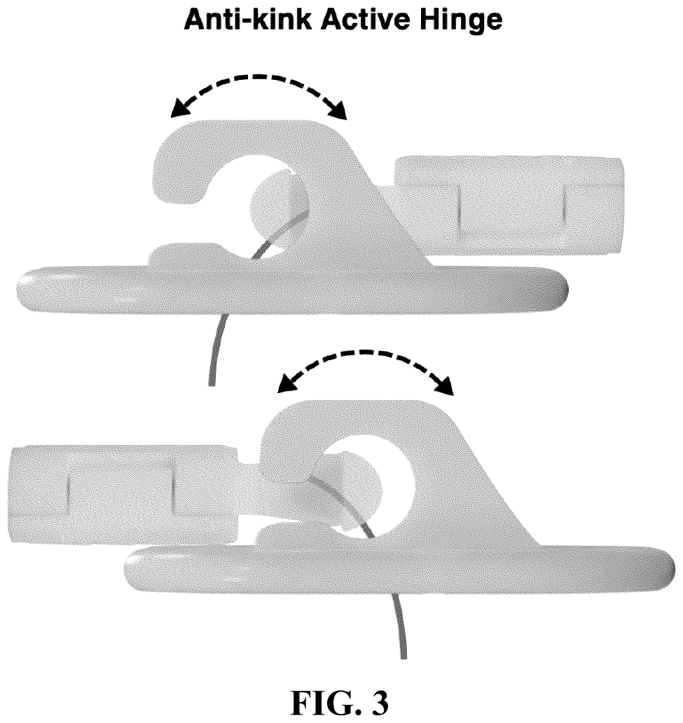

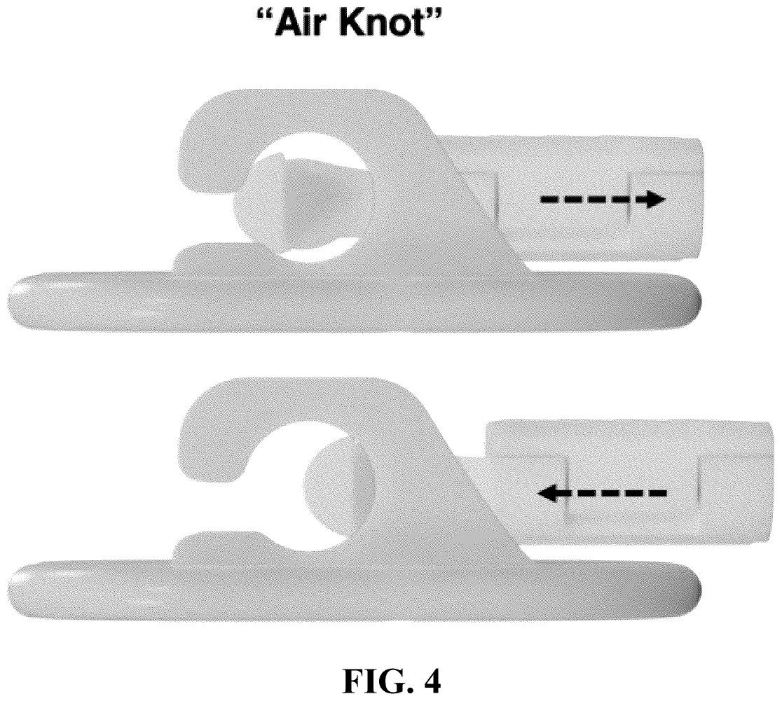

The hooks may be flexible or have a hinge to allow the struts to snap in place. In other embodiments, the clamp and back plate may be configured to attach together in two or more orientations. The hooks and struts may be symmetric or reversible to allow this. In some embodiments, once secured together, the clamp is able to rotate in one plane such that there is a fixed curvilinear distance between the drain incision site and the drain-clamp attachment site as shown in FIG. 3. Additionally, when assembled, the device may only allow for minimal in-and-out movement of the drain while keeping the drain firmly fixated to skin thereby creating a suture-less air knot. This may be formed using an oversized hook that allows for some movement of the strut within a confined physical space, as shown in FIG. 4. In the presently depicted embodiment, the inner portion of the hook is sized larger than the outer periphery of the strut allowing some lateral motion while still restraining the strut within the inner portion of the hook.

In the embodiment shown in FIGS. 6 and 7, the triangular grooves on the horizontal struts and the corresponding triangular rails on the back plate hooks create a keyed connection system that enforces a single, specific orientation for both connection and disconnection. When connecting the clamp to the back plate, the user must first visually align the triangular grooves with the triangular rails, then slide the struts into the hooks. The triangular profile creates a self-guiding effect, drawing the components into precise alignment as they engage. Similarly, to disconnect the components, the user must specifically orient the clamp to align the grooves with the rails before the struts can slide out of the hooks. This mechanism provides tactile feedback to healthcare providers during assembly and disassembly, confirming proper connection while substantially reducing the likelihood of accidental detachment during normal patient activities such as movement, coughing, or sneezing.

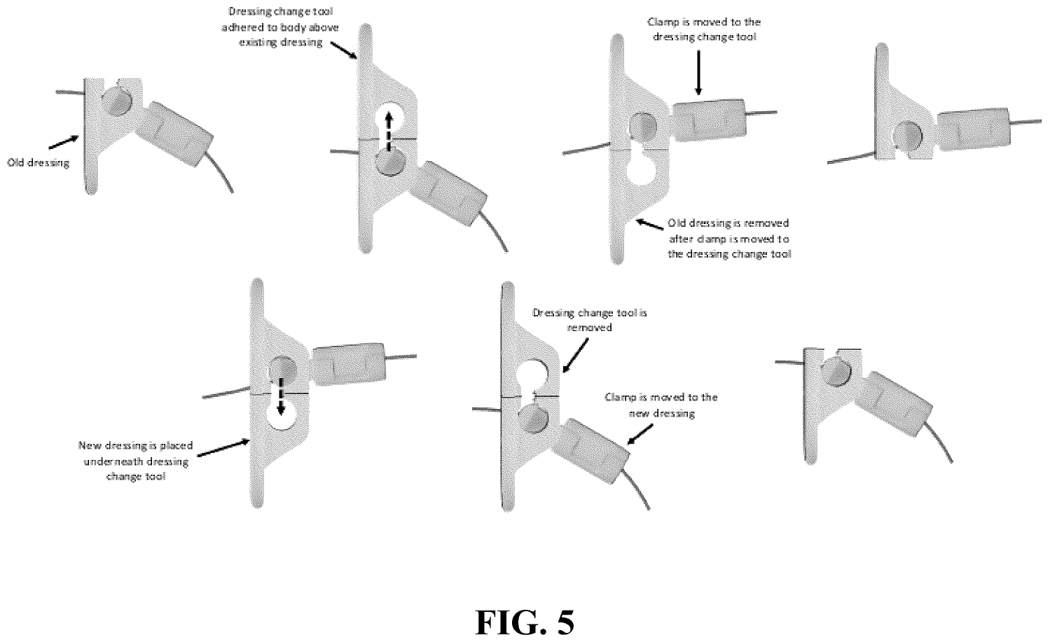

Referring to FIG. 5, the device may also include a dressing change tool. In some embodiments, the dressing tool is similar to the back plate in that it may include an adhesive material on its posterior side allowing for it to stick to skin. In some embodiments, the dressing change tool has hooks that stick out orthogonally from the bandage. These hooks may be similar in shape, size, and material to the hooks on the back plate. The horizontal struts on the securement device may be sized and configured to snap into the hooks on the dressing change tool in some embodiments. Similarly to the back plate, the struts may be sized and configured such that only one orientation of the clamp allows for the components to be snapped together and only one orientation of the clamp and dressing change tool allows for the two components to separate. The dressing change tool hooks may face the same direction as the back plate hooks or may face in a different or opposite direction to facilitate transfer of the clamp. In alternative embodiments, the dressing change tool may have a different number, type, or configuration of connectors for engaging the clamp. For example, the dressing change tool may have rings or slots for the clamp struts to slide into.

FIG. 5 also provides an exemplary method in accordance with the present disclosure. First, a drain may be fixed in place with a clamp and a backplate as described. Second, at the time of a dressing change, a dressing change tool may be adhered to the body above the existing dressing, such that the opening of the hooks on each component are in line with one another. Third, the clamp may then be transferred to the dressing change tool such that the curvilinear distance between the drain incision site and the clamp-drain junction remains constant (this maneuver does not malposition or dislodge the drain). This may be accomplished by squeezing the clamp or back plate to disengage the strut ends from the hook necks, and sliding the clamp upward until the struts engage the dressing change tool hooks. Fourth, the old dressing may be removed and replaced with a new dressing. Afterward, the clamp may subsequently be transferred to the new dressing and the dressing change tool removed by sliding the clamp down from the dressing change tool hooks onto the back plate hooks. In alternative embodiments, steps may be added, removed, or altered from this exemplary method. For example, the dressing change tool may not be used and the clamp may be disconnected entirely from the back plate to change the dressing.

M any embodiments of the present disclosure have notable advantages. The drain securement device does not evert or tug at skin during normal corporeal movements (breathing, sneezing, coughing, etc.), making it more comfortable for patients. The drain's dressing may be changed with ease, mitigating the need to cut gauze and tape to collect peri-catheter leakage. Further, because the tip of the drain is sized and configured to move freely in one plane about its hinge, the drain may have decreased intra-corporeal “play” (i.e. the end of the drain in the body should move around less when the outside tip is manipulated). This decreases the risk of dislodgement. Similarly, because the tip of the drain is sized and configured to move freely in one plane about its hinge at a fixed curvilinear distance from the drain incision site, the risk of catheter kinking with manipulation of the distal drain tip is reduced. This decreases the rate of drain occlusion.

Additionally, the triangular rail and groove system described herein provides enhanced security against accidental decoupling while maintaining ease of intentional connection and disconnection for healthcare providers. The keyed connection system ensures that the clamp remains securely attached to the back plate during normal patient movements and activities, yet can be readily disconnected when properly aligned by healthcare professionals during dressing changes or drain adjustments. This feature further reduces the risk of inadvertent drain dislodgement while maintaining the convenience and efficiency of the sutureless securement system.

What is claimed is:

1. A drain securement device comprising:
a drain engagement element comprising:
a cylindrical lumen running vertically through the drain engagement element and adapted to receive a drain, and a pair of horizontal struts extending from a top portion of the drain engagement element; and
a back plate comprising:
an adhesive material on a posterior surface adapted to adhere to a patient's skin; and
a pair of hooks configured to releasably engage the horizontal struts of the clamp;
wherein when the horizontal struts are engaged with the hooks, the clamp is fixed to the patient's skin while allowing rotation of the clamp in one plane to maintain a fixed curvilinear distance between a drain incision site and a drain-clamp attachment site.

2. The drain securement device of claim 1, wherein the drain engagement element comprises a front half and a back half that are configured to engage each other to form the cylindrical lumen.

3. The drain securement device of claim 2, wherein the front half and a back half are joined on one side by a hinge.

4. The drain securement device of claim 1, wherein the drain engagement element further comprises a protrusion extending into the lumen, wherein the protrusions sized and configured to clamp the drain without occluding the lumen.

5. The drain securement device of claim 1, wherein the horizontal struts and the hooks are configured to permit engagement and disengagement of the clamp with the back plate in only one orientation.

6. The drain securement device of claim 1, wherein, when the horizontal struts are engaged with the hooks, the drain engagement element allows for minimal longitudinal movement of the drain while maintaining the drain in a fixed position relative to the patient's skin.

7. The drain securement device of claim 1, further comprising a dressing change tool comprising: an adhesive material on a posterior surface adapted to adhere to the patient's skin; and a pair of hooks configured to releasably engage the horizontal struts of the drain engagement element.

8. The drain securement device of claim 7, wherein the hooks of the dressing change tool are aligned with the hooks of the back plate when the dressing change tool is adhered to the patient's skin above the back plate.

9. The drain securement device of claim 3, wherein the hinge is a book flap hinge.

10. A method of changing a dressing on a percutaneous drain fixed to a patient's skin, the method comprising:
adhering a dressing change tool to the patient's skin above a back plate including a pair of hooks, the dressing change tool comprising:
an adhesive material on a posterior surface; and
a pair of hooks on an anterior surface, the hooks aligned with the hooks of the back plate;
disengaging the horizontal struts of a drain engagement element from the hooks of the back plate and engaging the horizontal struts with the hooks of the dressing change tool;
removing the back plate from the patient's skin;
adhering a new back plate to the patient's skin;
disengaging the horizontal struts of the drain engagement element from the hooks of the dressing change tool and engaging the horizontal struts with hooks of the new back plate; and
removing the dressing change tool from the patient's skin;
wherein the fixed curvilinear distance between the drain incision site and the drain engagement element attachment site is maintained while the horizontal struts are engaged with the hooks of the dressing tool.

11. The method of claim 10, wherein the percutaneous drain is selected from the group consisting of a Jackson-Pratt drain, a Blake drain, a Penrose drain, and a pigtail catheter.

12. The method of claim 10, wherein the percutaneous drain is adapted to drain fluid from a body cavity selected from the group consisting of a pleural space, a peritoneal cavity, and a retroperitoneal space.

13. The method of claim 10, wherein securing the percutaneous drain with the drain securement device reduces at least one complication selected from the group consisting of drain dislodgement, drain kinking, and drain occlusion, as compared to securing the drain with sutures or adhesive alone.

14. The drain securement device of claim 1, wherein the horizontal struts comprise triangular grooves formed on outer surfaces of the struts.

15. The drain securement device of claim 14, wherein the hooks of the back plate comprise triangular rails protruding inwardly from inner surfaces of the hooks, the triangular rails configured to mate with the triangular grooves of the horizontal struts.

16. The drain securement device of claim 15, wherein the triangular grooves and triangular rails form a keyed connection that permits engagement and disengagement of the drain engagement element with the back plate only when the triangular grooves are aligned with the triangular rails.

17. The drain securement device of claim 16, wherein the keyed connection provides tactile feedback to confirm proper connection between the drain engagement element and the back plate.

18. The drain securement device of claim 16, wherein the keyed connection reduces risk of accidental decoupling during patient movement.

19. The drain securement device of claim 7, wherein: the horizontal struts comprise triangular grooves formed on outer surfaces of the struts; the hooks of the back plate comprise first triangular rails protruding inwardly from inner surfaces of the hooks; and the hooks of the dressing change tool comprise second triangular rails protruding inwardly from inner surfaces of the hooks, wherein both the first and second triangular rails are configured to mate with the triangular grooves of the horizontal struts.

20. A drain securement device comprising:
a drain engagement element comprising:
a lumen adapted to receive a drain; and
at least one horizontal strut extending from the drain engagement element, the at least one horizontal strut having a triangular groove formed on an outer surface;
a back plate comprising:
an adhesive material on a posterior surface adapted to adhere to a patient's skin; and
at least one hook configured to releasably engage the at least one horizontal strut, the at least one hook having a triangular rail protruding inwardly from an inner surface configured to mate with the triangular groove of the at least one horizontal strut;
wherein the triangular groove and triangular rail form a keyed connection that permits engagement and disengagement of the drain engagement element with the back plate only when the triangular groove is aligned with the triangular rail.
